(19) United States
(12) Patent Application Publication
(10) Pub. No.: US 2015/0094259 A1
(43) Pub. Date: Apr. 2, 2015

(54) Transdermal Delivery Devices

(71) Applicant: University Of Kwazulu-Natal, Westville (ZA)

(72) Inventors: Cephas Musabayane, Pinetown (ZA); Fanie Retief Van Heerden, Pietermaritzburg (ZA); Samson Mukaratirwa, Durban (ZA); Mark Andrew Tufts, Amansimtoti (ZA)

(73) Assignee: University Of Kwazulu-Natal, Westville (ZA)

(21) Appl. No.: 14/384,477

(22) PCT Filed: Mar. 7, 2013
(86) PCT No.: PCT/IB2013/051813

§ 371 Date: Sep. 11, 2014

(30) Foreign Application Priority Data
Mar. 13, 2012 (ZA) 2012/01838

(51) Int. Cl. A61K 38/28; A61K 9/00; A61K 47/36; A61K 31/7076

(52) U.S. Cl. CPC A61K 38/28; A61K 31/7076; A61K 9/0014; A61K 47/36
USPC 514/6.5; 514/5.9

(57) ABSTRACT

A transdermal delivery device in the form of a transdermal delivery patch for the delivery of insulin is disclosed. The patch comprises cross-linked amidated low methoxy pectin, insulin and a transdermal transfer enhancing agent.

THIS INVENTION relates to transdermal delivery devices. It relates, in particular, to transdermal delivery devices for the transdermal delivery of insulin.

Approximately 135 million people currently have diabetes mellitus. This figure is expected to increase to 300 million by the year 2025 in view of projected increases (170%) in developing countries and (40%) in developed countries [1]. Insulin is the standard pharmaceutical compound used in the treatment of diabetes and insulin is generally delivered intravenously (iv), intramuscularly (im) or subcutaneously (sc) and the injectable route is the most common method of administration. However, needle phobia and the stress associated with multiple daily injections which cause discomfort and inconvenience has given rise to a need for a less stressful route of insulin administration [2].

Transdermal drug delivery systems offer slow controlled release of drugs, avoid hepatic first pass metabolism, maintain constant blood levels for longer periods of time and decrease side effects thereby improving compliance. Since 1990, many investigations have been carried out to improve the transdermal delivery of insulin [3-8]. Methods that have proved useful include electroporation [3], lipid enhanced electroporation [3], topically applied biphasic vesicles [4], ultradeformable carriers [5], ultrasound [6] and microneedles [7]. An investigation has also highlighted the need for effective skin preparation and electrical enhancement [8]. However, all of these studies have made use of either chemical permeators or electrical impulses or sound waves to enhance delivery and none of them have used a transdermal delivery patch alone. The Applicant has now found that pectin can be used to transdermally deliver insulin.

According to a first aspect of the invention, there is provided a transdermal delivery device for the transdermal delivery of insulin, the device being in the form of a transdermal delivery patch comprising cross-linked amidated low methoxy pectin, insulin and a transdermal transfer enhancing agent.

The transdermal transfer enhancing agent may be dimethylsulphoxide, sodium oleate, sodium dodecyl sulphate (SDS or NaDS). The patch may comprise an antioxidant. It may, further, comprise an antibiotic.

The antioxidant may be Vitamin E, optionally combined with eucalyptus oil.

The antibiotic may be purmycin.

The amidated low methoxy pectin may have a degree of methoxylation of between about 19 and about 23 and a degree of amidation of between about 24 to about 31. Preferably the degree of methoxylation will be between 19 and 23 and the degree of amidation will be between 24 and 31.

The transdermal patch may be as herein described.

According to a second aspect of the invention, there is provided a method of treating diabetes, the method including applying a transdermal delivery patch comprising, cross-linked amidated low methoxy pectin, insulin and a transdermal transfer enhancing agent to the skin of a person or animal to deliver insulin through the skin of the person or animal.

The patch may be as hereinbefore described.

The invention thus provides a patch and a method for delivering insulin through the skin using a small conventional medicated skin patch. The components of the skin patch of the invention allow insulin to be transferred directly through the skin into the bloodstream. Transdermal delivery of insulin is known to be hindered by the fact that large molecule drugs, such as insulin, are not readily able to permeate the skin and therefore cannot enter the blood. The components of the skin patch of the invention overcome this problem by the incorporation of chemical enhancers which facilitate passage of unmodified insulin through the skin. Currently there are no approved insulin patches using transdermal delivery mechanisms for the delivery of insulation.

The invention is now described, by way of example, with reference to the following Examples and the Figures, in which

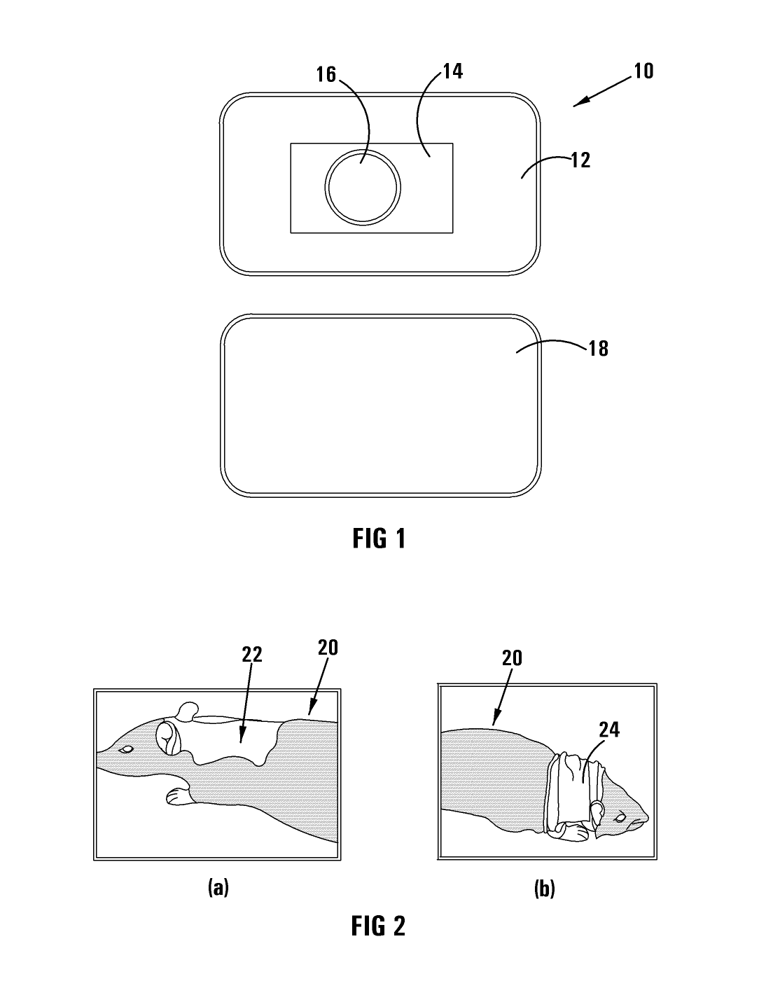

FIG. 1 shows a schematic diagram of two parts of a patch in accordance with the invention;

FIG. 2 shows the administration of the amidated pectin insulin matrix patch of the invention; and

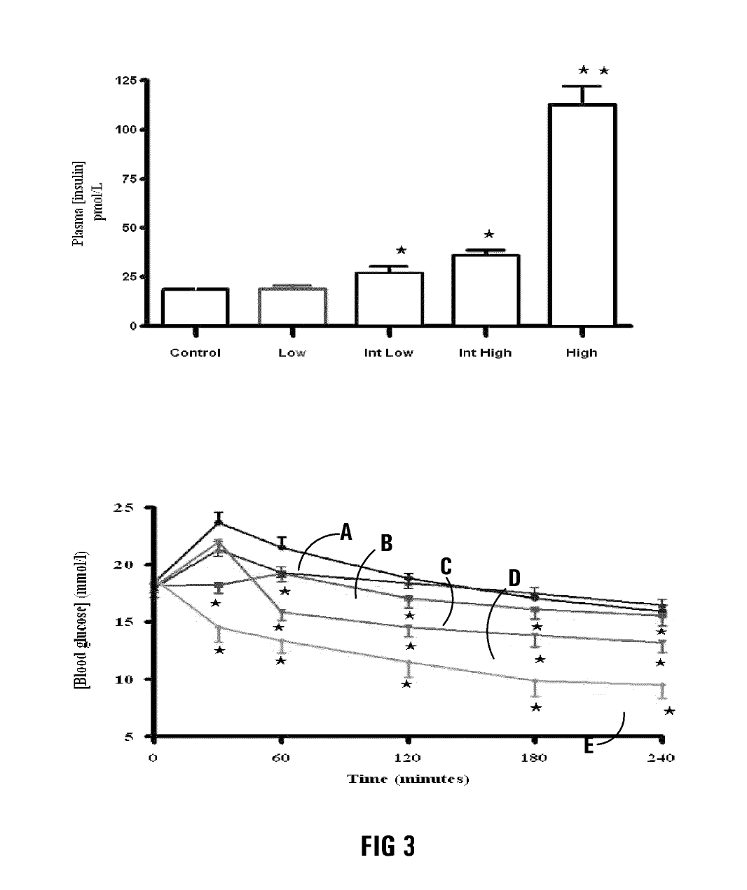

FIG. 3 shows a comparison of oral glucose tolerance (OGT) responses of streptozotocin (STZ)-induced diabetic rats to various doses of insulin in a pectin hydrogel patch with control animals; values are presented as means, and vertical bars indicate SEM of means (n=6 in each group); ★p<0.05 by comparison with control animals; ★★p<0.05 by comparison with all groups; A (control), B (low), C (int. low), D (int. high) and E (high).

FIG. 1 shows an embodiment of the transdermal delivery device of the invention in the form of a transdermal patch 10. The patch 10 is rectangular in shape and is 120 mm long and 100 mm wide. It comprises a hydrofilm backing 12 with a centrally located gauze strip 14 which is 80 mm long and 50 mm wide on the backing 12. A circular gel body 16 with a diameter of 25 mm comprising cross-linked amidated low methoxy pectin with a degree of methoxylation of 23 and a degree of amidation of 24, human insulin, dimethyl sulphoxide and vitamin E is centrally located on the gauze strip 14. The patch 10 is provided with an adhesive cover 18 (shown separately in the drawing).

FIG. 2 (a) schematically shows the application of the patch 10 to a rat 20. The back of the neck 22 of the rat 20 is smoothly shaved and the patch 10 is applied to the shaved area. FIG. 2 (b) shows the patch 10 secured in position with a jacket 24.

### EXAMPLES

### Insulin Patch Preparation

Materials and Methods

Drugs: Biphasic insulin (Actraphane HM, Novo Nordisk, Canada) or human insulin (Isophane Human Insulin, Lilly France SA, Fegershiem).

Matrix: Amidated low methoxy pectin with a degree of methoxylation of 23 and degree of amidation of 24.

Cross linking cations: Calcium chloride.

Penetration enhancers: Dimethyl sulphoxide.

Adhesive labels: Adhesive bandages or Hydrofilm (5 cm×7.5 cm; 8 cm×12 cm; 10 cm×20 cm.

Antioxidants: Vitamin E.

### Example 1

Amidated low methoxy pectin with a degree of methoxylation of 23 and degree of amidation of 24 was dissolved in deionized water (4 g/100 ml) to which various doses of Human insulin (6, 15, 30 and 60 μg) were added and mixed with agitation using a mixer (Heidolph laboratory mixer, Germany). Subsequently, dimethyl sulphoxide (3 ml) and vitamin E (3 ml) were added. This solution was mixed for a time period of 6 (six) hours. Following this, an aliquot of the mixture (10 ml) was transferred to a petri dish (424.62 cm²) and frozen at −5° C. After freezing, a 2% CaCl₂ solution was added on top of the frozen pectin and left to stand at room temperature for 10 minutes to allow for cross-linking and hence formation of the matrix patch. Patches with measured widths were cut out and placed on hydrofilm that served as backing material. The patches were stored at 2° C. in a refrigerator until use.

### Example 2

The following variations of the method of Example 1 were carried out.

### Example 2.1

Drugs: NovoRapid insulin (NovoRapid FlexPen, Novo Nordisk, Canada).

Matrix: Amidated low methoxy pectin with a degree of methoxylation of 19 and degree of amidation of 31.

Cross linking cations: Calcium chloride.

Penetration enhancers: Dimethyl sulphoxide.

Adhesive labels: Adhesive bandages.

Antioxidants: Vitamin E, Eucalyptus oil.

Antibiotic: Purmycin.

### Example 2.2

Drugs: Human insulin (NovoRapid FlexPen, Novo Nordisk, Canada)

Matrix: Amidated low methoxy pectin with a degree of methoxylation of 19 and degree of amidation of 31

Cross linking cations: Calcium chloride

Penetration enhancers: Sodium oleate

Adhesive labels: Adhesive bandages

Antioxidants: Vitamin E, Eucalyptus oil

Antibiotic: Purmycin

### Example 2.3

Drugs: Human insulin (NovoRapid FlexPen, Novo Nordisk, Canada)

Matrix: Amidated low methoxy pectin with a degree of methoxylation of 19 and degree of amidation of 31

Cross linking cations: Calcium chloride

Penetration enhancers: Sodium dodecyl sulfate (SDS or NaDS)

Adhesive labels: Adhesive bandages

Antioxidants: Vitamin E, Eucalyptus oil

Antibiotic: Purmycin

### Example 2.4

Drugs: Human insulin (NovoRapid FlexPen, Novo Nordisk, Canada)

Matrix: Amidated low methoxy pectin with a degree of methoxylation of 19 and degree of amidation of 31

Cross linking cations: Calcium chloride

Penetration enhancers: Dimethyl sulphoxide, Sodium oleate

Adhesive labels: Adhesive bandages

Antioxidants: Vitamin E, Eucalyptus oil

Antibiotic: Purmycin

### Example 2.5

Drugs: Human insulin (NovoRapid FlexPen, Novo Nordisk, Canada)

Matrix: Amidated low methoxy pectin with a degree of methoxylation of 19 and degree of amidation of 31

Cross linking cations: Calcium chloride

Penetration enhancers: Dimethyl sulphoxide, Sodium dodecyl sulfate (SDS or NaDS)

Adhesive labels: Adhesive bandages

Antioxidants: Vitamin E, Eucalyptus oil

Antibiotic: Purmycin

### Example 2.6

Drugs: Human insulin (NovoRapid FlexPen, Novo Nordisk, Canada)

Matrix: Amidated low methoxy pectin with a degree of methoxylation of 19 and degree of amidation of 31

Cross linking cations: Calcium chloride

Penetration enhancers: Sodium dodecyl sulfate (SDS or NaDS),

Sodium oleate

Adhesive labels: Adhesive bandages

Antioxidants: Vitamin E, Eucalyptus oil

Antibiotic: Purmycin

### Example 3

Amidated low methoxy pectin with a degree of methoxylation of 19 and degree of amidation of 31 was dissolved in deionized water (4 g/100 ml) with agitation using a mixer (Heidolph laboratory stirrer, Germany). Subsequently, either dimethyl sulphoxide, SDS or sodium oleate was added into the mixture. Vitamin E, eucalyptus oil and purmycin were added and mixed with agitation for 30 minutes. Various doses of NovoRapid insulin (4, 6, 8 and 10 units) were added to the mixture in the last 15 minutes of the preparation. Following this, an aliquot of the mixture (11 ml) was transferred to a petri dish (424.62 cm²) and frozen at −5° C. After freezing, the frozen pectin was left to stand at room temperature for 15 minutes then a 2% CaCl₂ solution was added onto the patch to allow for cross-linking and hence formation of the matrix patch. The patches were stored at 2° C. in a refrigerator until use.

### Transdermal Delivery of Insulin

Animals

Male Sprague-Dawley rats (90-300 g body weight) bred and maintained at Biomedical Research Unit, University of KwaZulu-Natal were used. The animals had free access to standard rat chow (Meadows, Pietermaritzburg, South Africa) and water, with a 12 h light/12 h dark cycle. Procedures involving animals and their care were conducted in conformity with institutional guidelines of the University of KwaZulu-Natal (Ethical Clearance 007/10/animal).

Male Sprague-Dawley rats (250-300 g) housed at the Biomedical Resource animal unit on the Westville campus of the University of Kwa-Zulu Natal were used in the study.

Determination of the Amount of Drug in Patches

In order to ascertain the amount of drug that was incorporated into the patches, the insulin content was determined in patches of known areas. Patches containing various doses of insulin were dissolved in Sorenson's phosphate buffer at a pH of 7.2. The amount of insulin added to the Petri dishes for each group was 0.6; 1.5; 3.0 and 6.0 μg respectively. This equated to a theoretical amount of 0.027; 0.08; 0.135 and 0.27 μg of insulin added to each patch. Individual patches were dissolved in the buffer and serial dilutions were done in order to measure the amount of insulin that was incorporated into each patch.

Application of the Hydrogel Patch

Rats were shaved on the dorsal region of neck 1-2 days prior to the application of the insulin patches. The hydrofilm backing the insulin hyrogel matrix patch was cut to the size of the patch and placed onto an adhesive to allow easy transfer onto the animal. The patches were held in place by an adhesive hydrofilm (Hartman-Congo Inc, Rock Hill, South Carolina, USA) which were adjusted for the size of the animal (FIG. 2).

Induction of Experimental Diabetes Mellitus

Diabetes mellitus was induced in rats with a single intraperitoneal injection of streptozotocin (STZ, 60 mg/kg) dissolved in freshly prepared 0.1 M citrate buffer (pH 6.3). Control animals were injected with the vehicle. Animals that exhibited glucosuria after 24 h, tested by urine strips (Rapidmed Diagnostics, Sandton, South Africa) were considered diabetic. Blood glucose concentration of 20 mmol/l or above measured after one week was considered as a stable diabetic state before experimental procedures were commenced.

Experimental Design

Non-diabetic and STZ-induced diabetic rats were divided into separate groups for oral glucose tolerance (OGT) response studies (n=6 in each group).

Insulin Hydrogel Patch

OGT effects of amidated insulin pectin hydrogel matrix patch were examined in separate groups of non-diabetic and STZ-induced diabetic groups of rats in which the patch applied onto the shaved area of the skin on the back of the neck (FIG. 2). The control animals were sham treated with drug free pectin patches.

Oral Glucose Tolerance (OGT) Responses

OGT responses were evaluated in separate groups of non-diabetic and STZ-induced diabetic groups of rats in which the patch was applied to the shaved area of the skin on the back of the neck (FIG. 2). The rats were divided into the following groups: non-diabetic control, non-diabetic treated, STZ-induced diabetic control and STZ-induced diabetic treated rats (n=6 in each group). Briefly, separate groups of non-diabetic and STZ-induced diabetic rats were fasted overnight (18 h) followed by measuring blood glucose (time 0). Subsequently, OGT responses to topically applied insulin pectin hydrogel patches at various doses of insulin (0.06; 0.21; 0.32 and 0.73 μg·Kg⁻¹ b wt) were monitored. In the control group of animals there was sham application of drug free pectin hydrogel matrix patches. Blood glucose was measured using a glucometer (Bayer's Glucometer Elite® (Elite (Pty) Ltd, Health Care Division, Isando, South Africa) before glucose loading and at 30, 60, 120 and 180 and 240 minutes after glucose-loading.

Determination of Plasma Insulin

Rats were sacrificed 4 hours after the start of the oral glucose tolerance test by an inhalation overdose of halothane in an anaesthetic chamber. Blood samples were then taken by cardiac puncture and transferred to heparinised tubes which were immediately centrifuged at 3000 rpm at 4 degrees Celsius for 15 minutes to pellet blood cells. The supernatant (plasma) was aspirated using a Pasteur pipette. Plasma insulin concentrations were evaluated by ultrasensitive rat insulin ELISA kit (DRG Instruments GmBH, Marburg, Germany) with 100% cross reactivity with insulin lispro (Humalog® Eli Lilly). The immunoassay is a quantitative method for the determination of plasma insulin utilizing two monoclonal antibodies which, together, are specific for insulin. The lower limit of detection was 1.74 pmoll⁻¹. The intra- and inter-assay analytical coefficients of variation ranged from 4.4% to 5.5% and 4.7% to 8.9%, respectively.

Statistical Analysis

All data were expressed as means±standard error of means (S.E.M.). Statistical comparison of the differences between the control means and experimental groups was performed with GraphPad InStat Software (version 4.00, GraphPad Software, San Diego, Calif., USA), using one-way analysis of variance (ANOVA), followed by Tukey-Kramer multiple comparison test. A value of p<0.05 was considered significant.

Results

Dissolution Studies

Table 2 shows the amount of insulin in insulin-pectin hydrogel patches. The theoretical amount of insulin in each patch was calculated from the known amount of insulin added to petri dishes during patch preparation and the area of the patches cut out of the petri dishes. The insulin incorporation into each patch ranged from 70% to 81%.

TABLE 2
Results of insulin pectin hydrogel patch dissolution studies

| Insulin in | Theoretical | Actual | Dosage | |
|---|---|---|---|---|
| petri dish | insulin in | insulin in | μg · kg⁻¹ | % insulin |
| (μg) | patch (μg) | patch (μg) | b. wt | incorporation |
| Low 0.6 | 0.027 | 0.019 | 0.06 | 70 |
| Int. Low 1.5 | 0.08 | 0.062 | 0.21 | 75 |
| Int. High 3.0 | 0.135 | 0.095 | 0.32 | 70 |
| High 6.0 | 0.27 | 0.22 | 0.73 | 81 |

Glucose Tolerance Responses

FIG. 3 shows the blood glucose responses of 5 groups of diabetic rats (n=10) to an oral glucose load. The 5 different groups were untreated controls, and rats treated with 0.06; 0.21; 0.32 and 0.73 μg·kg⁻¹ of insulin in a pectin hydrogel patch. In order to simplify the results of the tests the four treatment groups have been referred to as low dose, intermediate low dose, intermediate high dose and high dose, respectively.

Treatment with the high dose of insulin resulted in a significantly (p<0.05) lower blood glucose concentration at all time points throughout the glucose tolerance test, compared with all other doses of insulin. No significant difference (p>0.05) in blood glucose responses was seen for the 2 lowest doses at all time points compared with the controls. A significant reduction (p<0.05) in blood glucose was seen in rats treated with the intermediate dose compared with the control and 2 lower dose groups.

Plasma Insulin Concentrations

The plasma insulin concentrations of streptozotocin (STZ)-induced diabetic rats treated with various doses of insulin in a pectin hydrogel patch measured 4 hours after the start of the glucose response test are shown in FIG. 3. Values are presented as means, and vertical bars indicate SEM of means (n=6 in each group). ★p<0.05 by comparison with control animals

No statistical difference (p>0.05) was seen in the plasma insulin concentrations between the low dose and the control dose. The plasma insulin concentrations were significantly (p<0.05) higher in all other animals vs. the control animals. The plasma insulin concentrations found in the animals treated with the high insulin dose were significantly higher (p<0.05) than those found in all the other groups.

### Discussion

The invention provides adhesive pectin hydrogel skin patches that can deliver insulin into the bloodstream with a concomitant reduction in plasma glucose concentration in STZ-induced diabetic rats. Transdermal drug delivery is non-invasive offering slow controlled release of drugs and reducing degradation in the stomach and liver. Drug formulations from the pharmaceutical industry have previously consisted of simple, fast-acting chemical compounds that are dispensed orally or as injectables. The use of the transdermal delivery of drugs is usually limited by low skin permeability and the present invention demonstrates the enhanced permeation of drugs through the skin. Insulin is used extensively and the worldwide emergence of diabetes mellitus provides a large market potential. Approximately 215 million people currently suffer from diabetes mellitus. The treatment of diabetes usually requires daily subcutaneous (sc) injections and transdermal insulin delivery will therefore free diabetic patients from daily injections at the same time improving patient compliance. The major difference between the pectin patch of the invention and previous transdermal delivery systems is that the patch of the invention has the ability to transport insulin through the skin without the use of any additional mechanisms

### References

1. Stvring H, Andersen M, Beck-Nielsen H, Green A and Vach W. (2007) Counting drugs to understand the disease: The case of measuring the diabetes epidemic. Population Health Metrics, 5:2.
2. Musabayane C T, Munjeri O, Bwititi P and Osim E E. (2000). Orally insulin administered, insulin-loaded amidated pectin hydrogel beads sustain plasma concentrations of insulin in streptozotocin-diabetic rats. Journal of Endocrinology: 164: 1-6.
3. Transdermal insulin delivery using lipid enhanced electroporation. Sen, Daly and Hui. Biochimica et Biophysica Acta (BBA)—Biomemebranes. 1564 (1), August 2002, 5-8.
4. Transdermal delivery of insulin from a novel biphasic lipid system in diabetic rats. King, M J; Badea, I; Solomon J; Kumar, P; Gaspar, K J and Foldvari, M. Diabetes Techol Ther. 4 (4), 2002: 479-88.
5. Transdermal drug delivery of insulin with ultradeformable carriers. Cevc, G. Clinical Pharmacokinetics. 2003; 42 (5): 461-74.
6. Noninvasive ultrasonic transdermal insulin delivery in rabbits using the light-weight cymbral array. Lee, S; Snyder, B; Newnham, R E and Smith N B. Diabetes Technology Therapy. 2004, 6 (6): 808-15.
7. Transdermal delivery of insulin using microneedles in vivo. Martano, W; Davis, S P; Holiday, N R; Wang, J; Gill, H S and Prausnitz, M R. Pharmacology Research 2004 21 (6), 947-952
8. Transdermal delivery of regular insulin to chronic diabetic rats: effect of skin preparation and electrical enhancement. Zakzewski, C A; Wasilweski, J; Cawley, P and Ford, W. Journal of control release. 1998, 50 (1-3): 267-272.

1. A transdermal delivery device for the transdermal delivery of insulin, the device being in the form of a transdermal delivery patch comprising:
cross-linked amidated low methoxy pectin;
insulin; and
a transdermal transfer enhancing agent.

2. A transdermal delivery device as claimed in claim 1, in which the amidated low methoxy pectin has a degree of methoxylation of between about 19 and about 23 and a degree of amidation of between about 24 and about 31.

3. A transdermal delivery device as claimed in claim 1, in which the transdermal transfer enhancing agent is selected from dimethylsulphoxide, sodium oleate, sodium dodecyl sulphate and combinations of two or more thereof.

4. A transdermal delivery device as claimed in claim 1, in which the patch comprises an antioxidant.

5. A transdermal delivery device as claimed in claim 4, in which the antioxidant is vitamin E.

6. A transdermal delivery device as claimed in claim 4, in which the antioxidant is vitamin E in combination with eucalyptus oil.

7. A transdermal delivery device as claimed in claim 1, in which the patch further comprises an antibiotic.

8. A transdermal delivery device as claimed in claim 7, in which the antibiotic is purmycin.
